(19) United States
(12) Patent Application Publication
(10) Pub. No.: US 2009/0112217 A1
(43) Pub. Date: Apr. 30, 2009

(54) IMPLANT INSERTION DEVICE

(76) Inventor: Douglas Hester, Raynham, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/930,719

(22) Filed: Oct. 31, 2007

(51) Int. Cl. A61B 17/60

(52) U.S. Cl. 606/99

(57) ABSTRACT

The present invention is directed to a device for inserting orthopaedic implants, more specifically spinal implants such as artificial discs.

### BACKGROUND OF THE INVENTION

1. Field of the Invention

The present invention relates to implant insertion devices, such as for orthopaedic devices and more particularly to implant insertion devices for spinal implants.

2. Related Art

Devices for insertion of implants are known. U.S. Pat. No. 6,066,174 and U.S. Pat. No. 5,782,830 describe the use of a grabbing element which is integral to an insertion handle. A movable sleeve slides over the neck of the grabbing element. Through the use of interfering tapers, the grabbing element increases its grip on vertebral body spacer.

U.S. Pat. No. 6,159,215 describes the Acromed Keystone Inserter. This device also makes use of interfering tapers to generate a grip on a vertebral body spacer. In this case, the sleeve is held fixed to a front handle portion and the grabbing element is retracted using a rear handle portion. The rear handle is connected to the grabbing element so as to create a mechanical advantage in retracting it and consequently creating a grip on the implant.

U.S. Pat. No. 6,319,257B1 describes the use of a drive thread to create a mechanical advantage on the grabbing element. The grip is generated using interfering tapers.

### BRIEF DESCRIPTION OF THE DRAWINGS

FIGS. 1a-d depict a preferred embodiment of this invention.

FIGS. 2a-b depict an alternate and more general embodiment of this invention.

FIGS. 3a-c depict interactive aspects of the housing with the grabber tips for a preferred embodiment of the invention.

### SUMMARY OF THE INVENTION

One embodiment of this invention is directed toward a device for inserting an implant comprising:
a) a housing having a distal end and a proximal end;
b) a shaft having a distal end and a proximal end;
c) gripping tips for gripping the implants, the gripping tips integrally formed at the distal end of the shaft, the gripping tips movable between an implant gripping position and an implant releasing position;
d) the shaft slidably movable within the housing for causing the gripping tips to grip and release the implant;
e) a spring for biasing the gripping tips to a gripping position, the spring contacting and biasing the shaft relative to the housing.

Another embodiment of this invention is directed toward a sterile implant system contained in a sterile package comprising:
a) a sterile implant; and
b) a sterile implant inserter attached to the implant.

Yet another embodiment relates to a method for manufacture of a sterile implant system comprising:
a) providing an implant;
b) providing an implant inserter;
c) attaching the implant to the inserter;
d) placing the implant with inserter in a package; and
e) terminally sterilizing the package containing the implant and inserter.

Preferably, the inserter is made of a radiolucent material.

### DETAILED DESCRIPTION OF PREFERRED EMBODIMENTS OF THE INVENTION

The present invention is a device for inserting a spinal implant. More specifically, this device is intended for use with artificial discs.

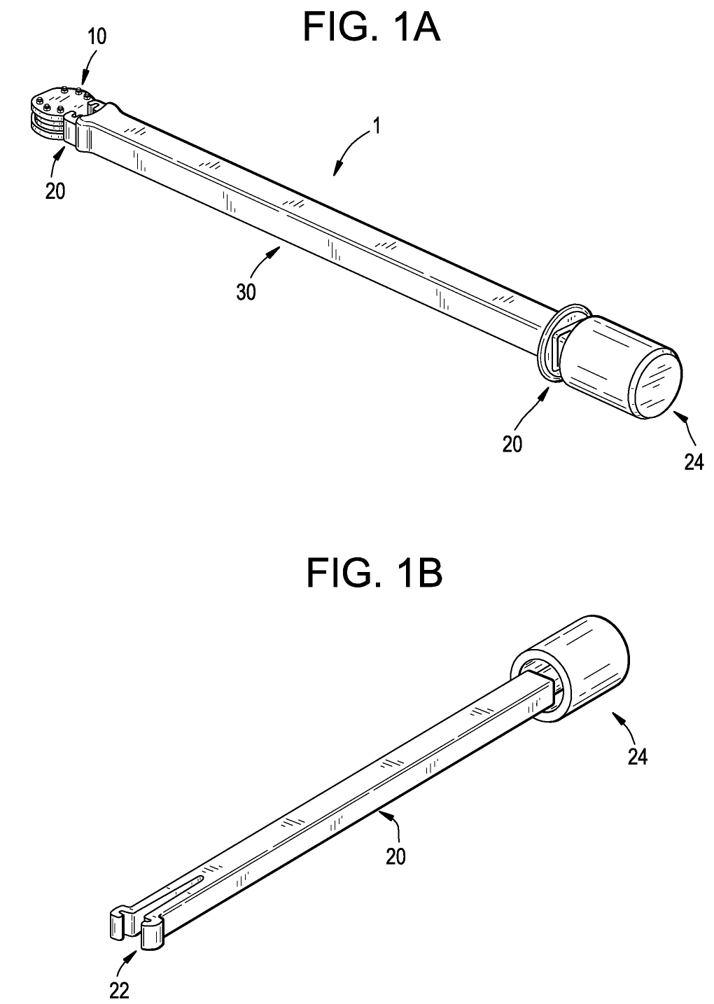

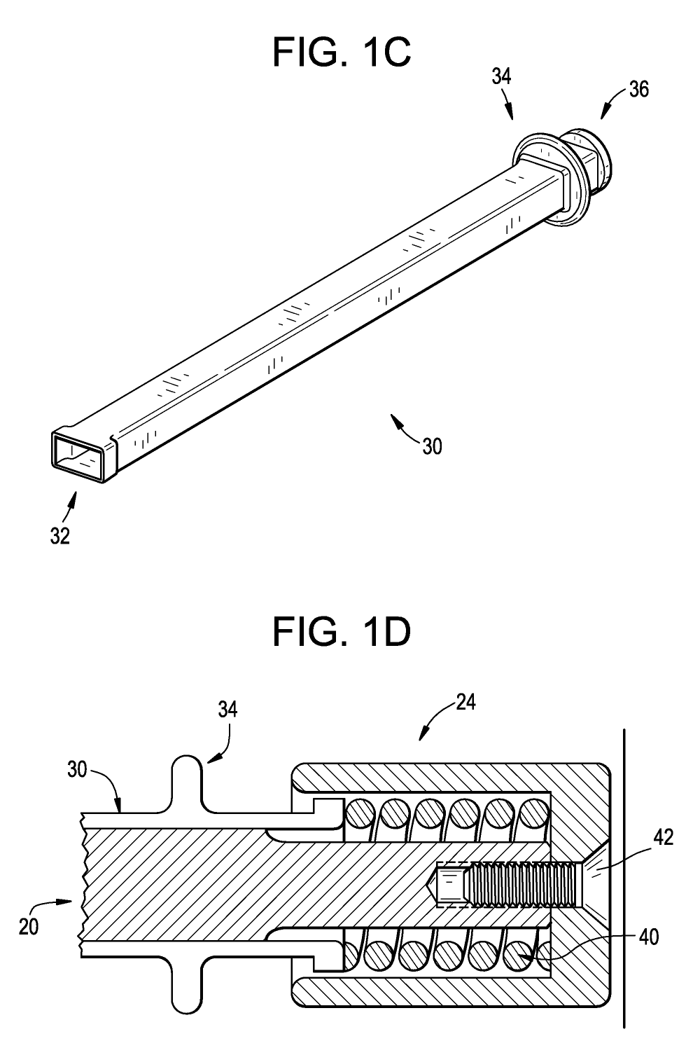

FIGS. 1a-d show inserting device 1 grasping an implant 10. Device 1 comprises housing 30, shaft 20 comprising grasping tips 22 located at the distal end of shaft 20 and handle 24 located at the proximal end 24 as shown in FIG. 1b. FIG. 1c shows housing 30 comprising a flared distal end 32, a grip 34 and proximal end 36. Shaft 20 slidably moves within housing 30. FIG. 1d shows details of handle 24 and its interaction with the shaft 20, namely wherein spring 40 exerts a biasing force between housing 30 and shaft 20 to bias gripping tips 22 to a grasping position of implant 10. Handle 24 is attached to shaft 20 such as by threaded fastener 42. In operation when handle 24 is urged toward grip 34, shaft 20 extends further beyond the distal end 32 of housing 30 and gripping tips 22 are flexed open, thereby releasing implant 10.

The inserting device can be packaged with the implant in the configuration shown in FIG. 1a. By packaging the implant in this way, an intermediate loading devices (such as clips) are not required. The inserter acts as the clip to hold the implant together, particularly when the implant is a multicomponent implant such as an artificial disc.

In use, inserting device 1 and implant 10 are first removed from sterile packaging. Holding the handle 24, the implant 10 is inserted into the cleared space of implantation. To detach the grabbing tips from the implant, the handle 24 is moved toward grip 34. The intended technique for release of the disc is analogous to depressing the plunger on a syringe.

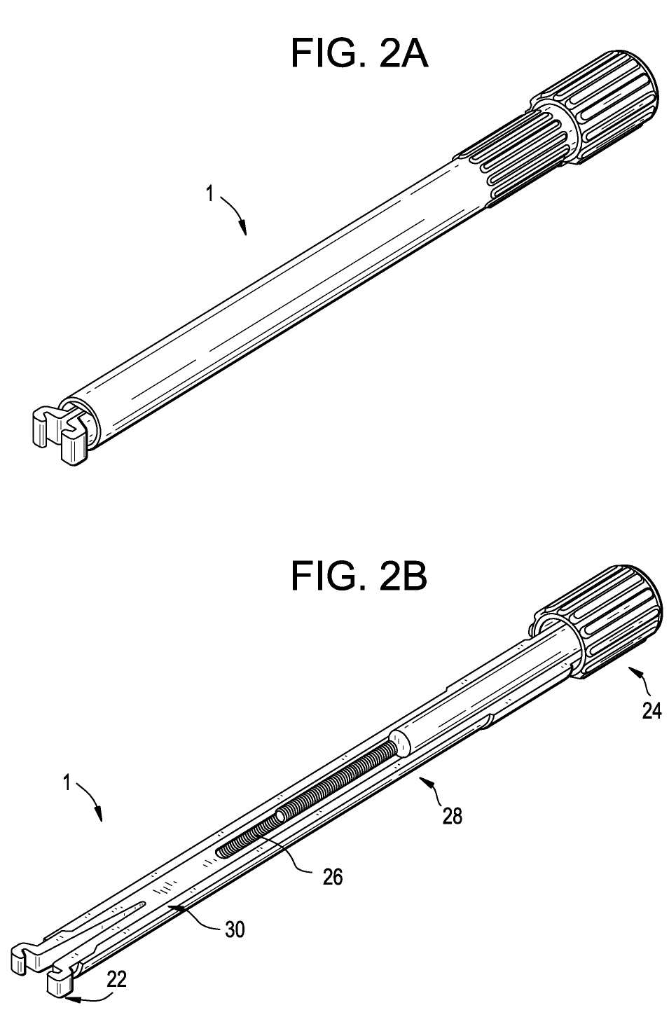

An alternate embodiment for the present invention is shown in FIGS. 2a and 2b. In this embodiment the direct mechanism of closing the grabber tips on the implant remains the same as the previously discussed embodiment. The housing is driven into the tapered neck of the grabber tips. In this case, the housing is driven into the tapered neck by rotating the handle 24 relative to the housing 30; in accomplishing this rotation, the male threads 28 of the proximal shaft engage the female threads 26 of the distal shaft thereby urging the distal shaft 22 into or out of relative engagement with the housing 30. The matched threads between the housing and inserter base shaft provide the necessary mechanical advantage.

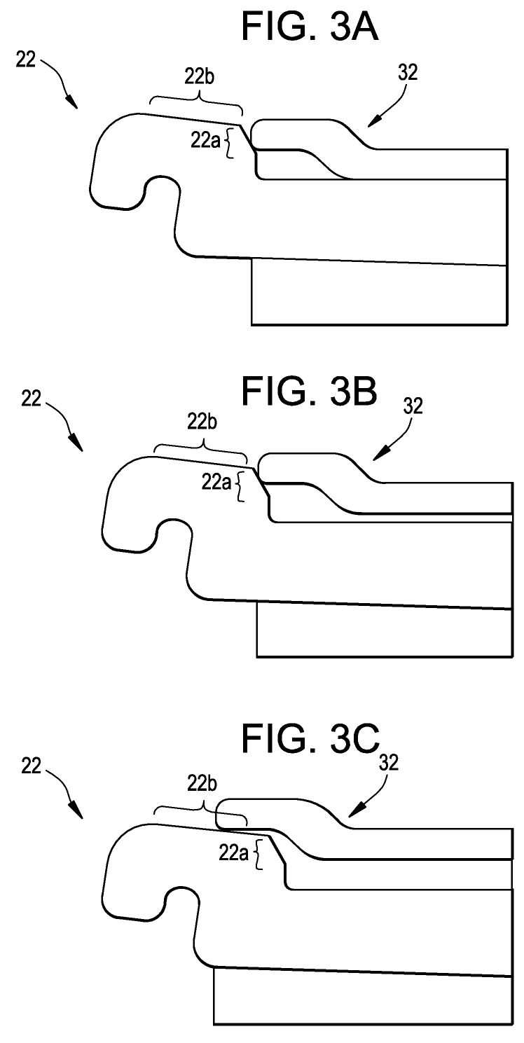

FIGS. 3a-c show a preferred feature of the device of this invention. This feature is directed to a dual tapered housing at the housing's distal end and corresponding dual tapered grasping tips. This feature allows for large mechanical advantage without the need for large axial travel of the gripping tips with respect to the housing whereas the prior art makes use of a single taper.

Referring to FIG. 3a, the first taper set 22a is steep for rapid closure and the second taper set 22b is shallow for mechanical advantage for adequately securing the implant.

The different positions involved in securing an implant are described below, namely the interaction of the grasping tips 22 with the housing opening 32.

FIG. 3a shows the open position of the device wherein the housing shaft 20 is fully extended by the user (by urging the handle toward the grip) thereby permitting the grasping tips 22 to open. This position is used prior to grasping and releasing the implant.

FIG. 3b shows the gripping or grasping position of the device wherein the handle is partially released and compressed spring 40 causes the housing 32 to move toward the grasping tips 22. The end of the housing 32 encounters the first steep taper 22a on the neck of the grasping tips 22. The grasping tips 22 close quickly with little change in housing 32 position/travel. The steeper taper angle of 22a provides less mechanical advantage in terms of closing the grasping tips 22 but, during this phase, the grasping tips 22 are not in firm contact with the implant and therefore little closing force is required.

FIG. 3c shows the gripping or grasping phase, wherein the grasping tips 22 are in firm contact with the implant. The neck of the housing 32 encounters the shallow taper 22b on the grasping tips 22. This shallow taper 22b allows a relatively weak spring to be used in generating the necessary forces to securely hold the implant. A weak spring allows the surgeon to move the housing (with respect to the grasping tips) without the need for mechanical advantage. This may reduce the complexity of the device.

It should be noted that if a single shallow taper were used, the amount of handle/spring travel would be excessively large. Therefore, the initial steep taper 22a is used to bring the grabber tips in contact with the implant. The shallow taper 22b engages only when a high compressive force is required to secure the implant.

In yet another embodiment, the invention is directed toward an implant system comprising an implant and an implant inserter. The system preferably is sterile and packaged sterile.

While the inserter may be made of any material of sufficient strength and rigidity suitable for medical use, a preferred embodiment includes the advantage or convenience of having an implant provided with a radiolucent inserter. These advantages include the ability to more precisely insert an implant wherein the radiolucent nature of the inserter does not obscure placement during fluoroscopic imaging or radiographic imaging (i.e., no need to remove the inserter as with conventional metal inserters); less likely to result in damage to the implant as radiolucent materials are predominately plastic; lightweight as compared to conventional metal inserters; and can be made disposable due to the relatively lower cost of injection molded components.

The inserter components may be made by injection molding techniques known in the art. Thus each element of the inserter, e.g., in referring to FIGS. 1a-d, the housing 30, shaft 20, handle 24, spring 40 are capable of being injection molded and easily assembled. For example, assembly of the inserter shown in FIGS. 1a-d, is accomplished by sliding housing 30 over shaft 20. With the proximal end of shaft 20 extending beyond the proximal end of housing 30, i.e., end 36, spring 40 is slid over the proximal end of shaft 20 and handle 24 is secured to shaft 20 to contain bias spring 20 between housing 30 and shaft 20. The biasing force of spring 20 causes the distal end of housing 30 to compress tips 22 of shaft 20 to grasp and secure implant 10. Securing of handle 24 to shaft 20 can be by snapping these parts together or by threading them together as shown.

Thus in a preferred embodiment, all of the components of the inserter are desirably made of radiolucent materials which are materials well known in the art and have the characteristics of being relatively penetrable by x-rays and other forms of radiation. It should be understood, however, that there may be embodiments wherein the user will only require the part of the inserter that will find itself in a fluoroscopic or radiographic field to be in made of a radiolucent material. Radiolucent materials allow for fluoroscopic or radiographic viewing which facilitates the precise placement of the implant, relatively unobscured as compared with radiopaque materials such as metals. Examples of suitable radiolucent materials include plastics and plastic composites. These materials are generally easy to extrude, well suited for compression molding techniques, and easily used in injection molding techniques. The plastic composites may include a reinforcement material for structural strength or rigidity. Such reinforcement materials include carbon fiber, glass, or fiberglass. The plastics can be thermoset or thermoplastic in nature.

For instances where the inserter will be reused by the hospital, the radiolucent material is preferably one that can be subjected to standard hospital sterilization procedures without substantial degradation. Polymers, copolymers and composites may be used. A heat resistant thermoplastic such as RADEL® polyarylethersulfone (PAES) may be used since it is understood to be sterilizable in a steam autoclave. RADEL® PAES is understood to be available from Amoco Polymers, Inc. of Alpharetta, Ga., and from suppliers such as Piedmont Plastics, Inc. of Charlotte, N.C. It may be desirable to use a different material that is more radiolucent. At least some commercially available acetal copolymers can be used, such as DELRIN® material available from E.I. DuPont de Nemours and Co. of Wilmington, Del. and CELCON® polyoxymethylene available from Celanese Corporation through Ticona-US of Summit, N.J. Also useful are acrylonitrile-butadiene-styrene (ABS) copolymers. In addition, aluminum can be used for some of the radiolucent materials. In some instances, the radiolucent material may be made of anodized aluminum. When hollow parts such a housing 30 are used as they should be adequately radiolucent. It should be understood that the present invention is not limited to any particular radiolucent material or to any particular degree of radiolucency unless expressly called for in the claims. Generally, use of the term “radiolucent” is intended herein to include any material that will allow the surgeon to view anatomic landmarks either fluoroscopically or radiographically.

Preferred radiolucent materials include RADEL® polyarylethersulfones, Delrin® acetal copolymers, polycarbonates, nylon, and acrylonitrile-butadiene-styrene (ABS) copolymers.

Example of implants that can be used with the inserters of this invention include spinal implants and other orthopaedic implants. Example of suitable spinal implants include vertebral body spacers (fusion cages and bone grafts), vertebral body replacements, and artificial discs. Suitable orthopaedic implants include bone grafts and bioabsorbable implants comprising bone anchors and screws.

Another embodiment of this invention is directed toward a sterile implant system contained in a sterile package comprising:
a) a sterile implant; and
b) a sterile implant inserter attached to the implant.
   In this embodiment the implant system is assembled in a sterile environment with the implant and inserter being provided as sterile or are sterilized in the sterile environment. Prior to packaging, the implant is attached to the inserter and sealed in the sterile package.

Yet another embodiment relates to a method for manufacture of a sterile implant system comprising:
a) providing an implant;
b) providing an implant inserter;
c) attaching the implant to the inserter;
d) placing the implant with inserter in a package; and
e) terminally sterilizing the package containing the implant and inserter.
   In this embodiment, the implant system may be assembled in an environment not classified as sterile (i.e. clean room). The implant is simply attached to the inserter and packaged. The entire packaged assembly is terminally sterilized by methods know in the art including but not limited to ethylene oxide, gas plasma, and radiation such as gamma or e-beam irradiation.

It should be understood that the foregoing disclosure and description of the present invention are illustrative and explanatory thereof and various changes in the size, shape and materials as well as in the description of the preferred embodiment may be made without departing from the spirit of the invention.

What is claimed is:

1. A device for inserting an implant comprising:
a) a housing having a distal end and a proximal end;
b) a shaft having a distal end and a proximal end;
c) gripping tips for gripping the implants, the gripping tips integrally formed at the distal end of the shaft, the gripping tips movable between an implant gripping position and an implant releasing position;
d) the shaft slidably movable within the housing for causing the gripping tips to grip and release the implant;
e) a spring for biasing the gripping tips to a gripping position, the spring contacting and biasing the shaft relative to the housing.

2. The device of claim 1, further comprising a handle attached to the proximal end of the shaft, the handle encompassing the proximal end of the housing, the handle sized to receive the spring which biases the shaft relative to the housing.

3. The device of claim 2, when in the housing further comprises a rim at the proximal end of the housing.

4. A sterile implant system contained in a sterile package comprising:
a) a sterile implant; and
b) a sterile implant inserter wherein the sterile implant is grasped by the sterile implant inserter.

5. The system of claim 4, wherein the inserter is made of a radiolucent material selected from the group consisting of thermoset plastics and thermoplastics.

6. The system of claim 5, wherein the radiolucent material is RADEL® polyarylethersulfone.

7. The system of claim 5, wherein the radiolucent material is Delrin® acetal copolymer.

8. The system of claim 5, wherein the radiolucent material is a polycarbonate.

9. The system of claim 5, wherein the radiolucent material is nylon.

10. The system of claim 5 wherein the radiolucent material is an acrylonitrile-butadiene-styrene (ABS) copolymer.

11. The system of claim 4, wherein the implant is selected from the group consisting of spinal and orthopaedic implants.

12. The system of claim 11, wherein the implant is a spinal artificial disc.

13. The system of claim 11, wherein the spinal implant is a vertebral body spacer.

14. The system of claim 13, wherein the spinal implant is a fusion cage.

15. The system of claim 13, wherein the spinal implant is a bone graft.

16. The system of claim 11, wherein the spinal implant is a vertebral body replacement.

17. The system of claim 11 wherein the orthopaedic implant is a bone graft.

18. The system of claim 11 wherein the orthopaedic implant is a bioresorbable implant comprising bone anchors and screws.

19. A method for manufacture of a sterile implant system comprising:
a) providing an implant;
b) providing an implant inserter;
c) attaching the implant to the inserter;
d) placing the implant with inserter in a package; and
e) terminally sterilizing the package containing the implant and inserter.

20. The method of claim 19, wherein the inserter is made of a radiolucent material selected from the group consisting of thermoset plastics and thermoplastics.

21. The method of claim 20, wherein the radiolucent material is RADEL® polyarylethersulfone.

22. The method of claim 20, wherein the radiolucent material is Delrin® acetal copolymer.

23. The method of claim 19, wherein the implant is selected from the group consisting of spinal and orthopaedic implants.

24. The method of claim 23, wherein the implant is a spinal artificial disc.

25. The method of claim 23, wherein the implant is a vertebral body spacer.
